(19) United States
(12) Patent Application Publication
(10) Pub. No.: US 2007/0286777 A1
(43) Pub. Date: Dec. 13, 2007

(54) Aura-Sphere

(76) Inventor: Victor Vincent Riley, South Bend, IN (US)

Correspondence Address: Victor V. Riley, 1009 East Fox Street, South Bend, IN, 46613, US

(21) Appl. No.: 11/335,887

(22) Filed: Jun. 7, 2006

(51) Int. Cl. A61L 9/03

(52) U.S. Cl. 422/125; 422/5

(57) ABSTRACT

Vitamin Mineral Aura is an Aura created by the proper use of my invention called the Aura-Sphere, APPL No. 29/251,646 Application: Placing lit vitamin candle within my invention (the Aura-Sphere) given 1 hour brings the Aura-Sphere to it's operating temperature. The Vitamin Candle feeds the cold absorbing granite Sphere. When Atoms are dormant within the granite they absorb. As the vitamin candle is heating the granite sphere, it is releasing it's vapor of Vitamin E into the absorbent Granite Sphere. The granite's core becomes warmer from the flame of the vitamin candle, starts a reverse cycle within the granite sphere and begins to release a mineral enriched vitamin aura. The utility function of my invention The Aura-Sphere creates a vitamin mineral aura that is absorbed through touch for it's therapeutic benefits!

The Aura-Sphere consisting of a metal stand that suspends one or more granite sphere above a candle. Different types of candles may be used. The heat from the candle warms the sphere, which radiate the thermal energy outward and can be touched for therapeutic value. Granite is made up of minerals: Plagioclase, Quartz, Potassium Feldspar, Muscovite, Biotite, Amphibole, Pyroxene, and Olivine. When Plutonic minerals with-in the Sphere are heated, a Kinetic Aura is formed by its atoms hitting each other. Sphere releasing its Plutonic mineral energy for therapy. Thus, the reason behind the title Aura Sphere.

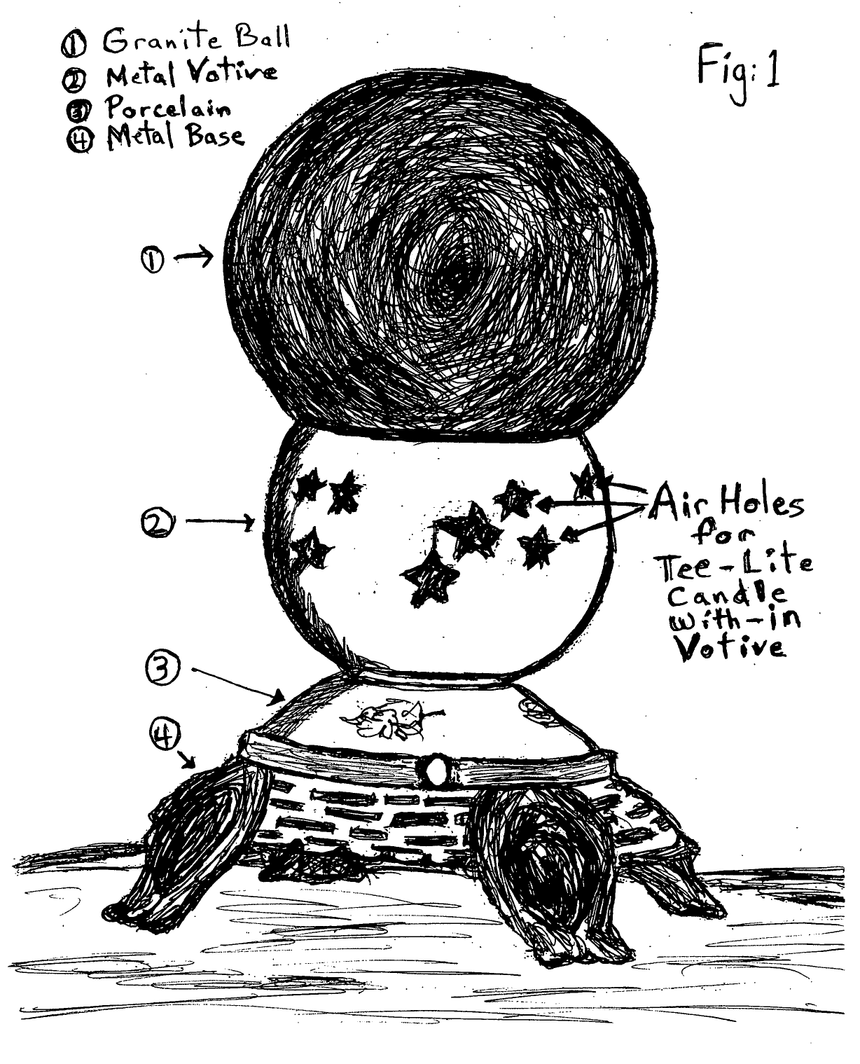

FIG. 1 is a side view drawing of a Aura-Sphere showing my original new design single sphere.

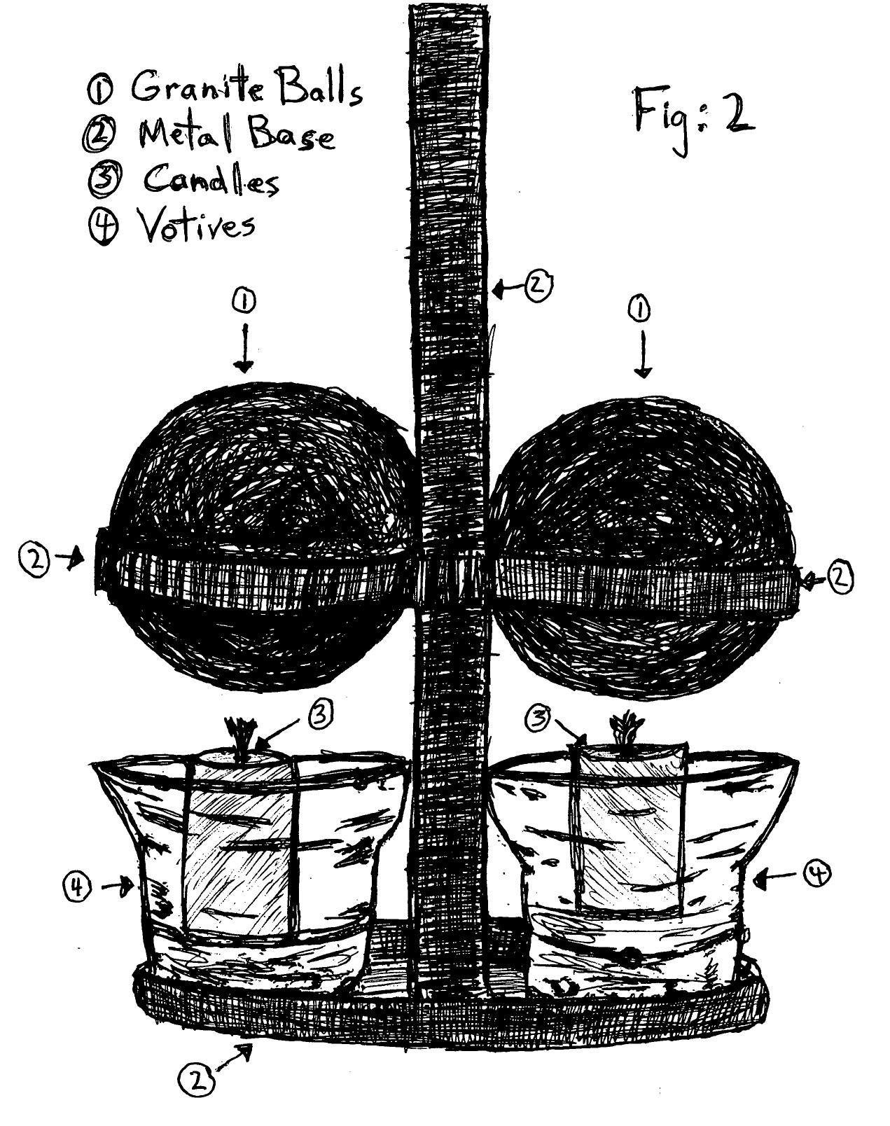

FIG. 2 is a side view drawing of a Aura-Sphere showing my original new design double sphere.

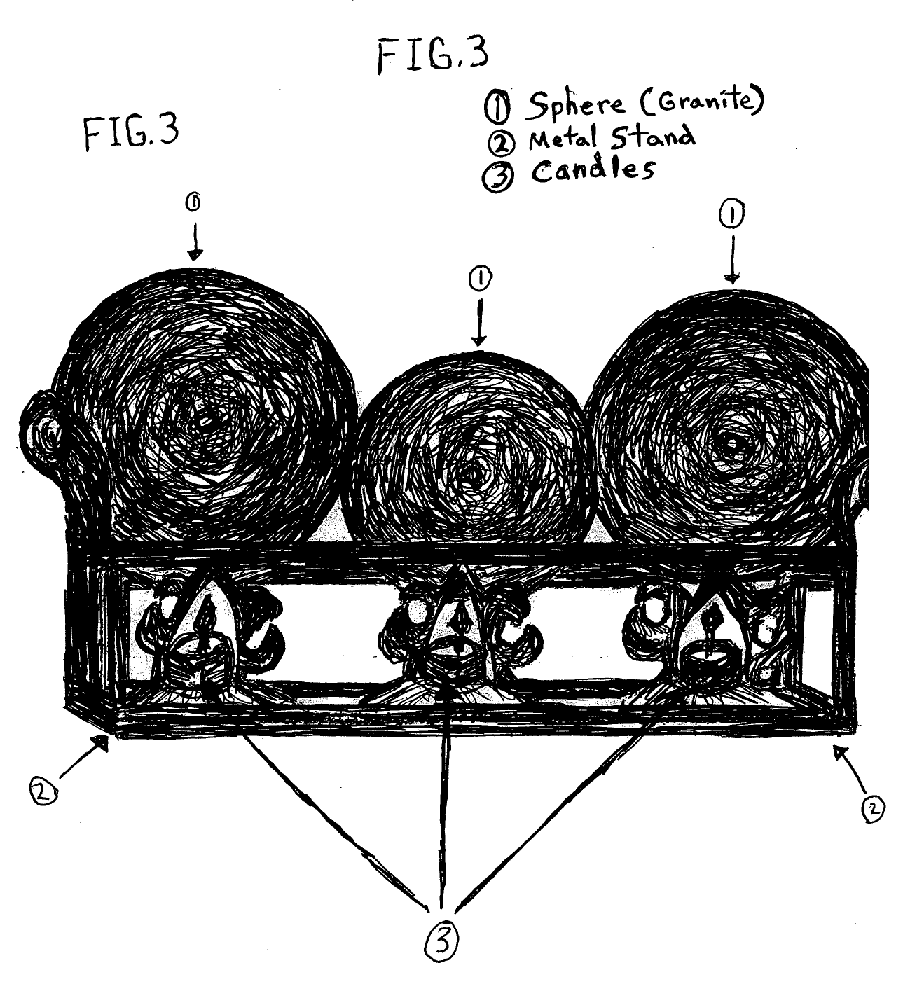

FIG. 3 is a side view drawing of a Aura-Sphere Showing my original new design triple sphere.

1. I claim: the function Utility for a Aura-Sphere as shown.
I claim: Utility of my Aura Sphere (FIG. 1) invention's properties of stimulation that with, the lit candle under the granite sphere radiates the thermal energy outward from the sphere and can be touched for therapeutic value.
I claim: Utility of my triple Aura Sphere (FIG. 3) invention's properties of stimulation with that of the lit candles under the granite spheres radiates the thermal energy outward from the spheres and can be touched for therapeutic value.
